(19) United States
(12) Patent Application Publication
(10) Pub. No.: US 2025/0360106 A1
(43) Pub. Date: Nov. 27, 2025

(54) PREVENTING IMMUNOTHERAPY-INDUCED EDEMA USING ANGIOTENSIN RECEPTOR BLOCKERS

(71) Applicant: The General Hospital Corporation, Boston, MA (US)

(72) Inventors: Rakesh K. JAIN, Wellesley, MA (US); Meenal DATTA, Notre Dame, IN (US); Mario SUVA, Boston, MA (US); Lei XU, Acton, MA (US)

(21) Appl. No.: 18/872,375

(22) PCT Filed: Jun. 9, 2023
(86) PCT No.: PCT/US2023/024963

§ 371 Date: Dec. 6, 2024

Related U.S. Application Data
(60) Provisional application No. 63/350,574, filed on Jun. 9, 2022.

(51) Int. Cl. A61K 31/4178; A61K 31/405; A61K 39/395; A61P 7/10

(52) U.S. Cl. CPC A61K 31/4178; A61K 31/405; A61K 39/3955; A61P 7/10

(57) ABSTRACT

The disclosure features methods and compositions for the prevention of immunotherapy induced edema in glioblastoma patients undergoing immunotherapy.

### CROSS-REFERENCE TO RELATED APPLICATIONS

This application claims priority to U.S. Provisional Application No. 63/350,574, filed Jun. 9, 2022, which is incorporated herein by reference in its entirety.

### STATEMENT REGARDING FEDERALLY SPONSORED RESEARCH

This invention was made with government support under R35-CA197743, U01-CA224348, RO1 CA259253, RO1 CA208205, and R01-NS118929 awarded by the National Institutes of Health. The government has certain rights in the invention.

### BACKGROUND

Glioblastoma (GBM), the most common primary brain tumor in adults, has a profoundly poor prognosis of less than 2 years overall survival (OS) even under currently available aggressive treatments. Despite reports that GBM can be cured in some animal models with immune checkpoint blockers (ICBs), this immunotherapeutic approach has failed in all phase III trials, has had only limited success in improving the OS of GBM patients in some phase II trials and may benefit less than 10% of all GBM patients. In addition to low mutational burden, poor antigenicity, and immunosuppressive tumor microenvironment (TME), a challenge unique to GBM is the brain edema exacerbated by anti-programmed death/ligand 1 (PD1/PD-L1) antibodies. Currently, this increased edema is controlled by potent steroids that are highly immunosuppressive, and thus compromises the benefit of ICBs.

It would be advantageous to have a treatment that could control or eliminate the ICB-induced edema in these patients. Indeed, improving immunotherapy outcomes for many glioblastoma patients remains a critically unmet need.

### SUMMARY OF THE INVENTION

Used in lieu of immunosuppressive corticosteroids, the angiotensin receptor blocker losartan prevented ICB-induced edema and reprogrammed the tumor microenvironment, curing 20% of mice which increased to 40% in combination with standard of care treatment.

Using single-cell RNA sequencing (scRNASeq), intravital imaging, and appropriate blocking strategies, we show that this immunotherapy induced edema is not mediated by canonical vasogenic mechanisms (e.g., VEGF overexpression), but rather by an inflammatory response to ICB treatment. We also show that the angiotensin receptor blocker (ARB) losartan overcomes ICB-induced edema by reducing endothelial membrane-type matrix metalloproteinase 1 and 2 (MT-MMP-1, -2) activity that is upregulated in response to ICB. Furthermore, losartan decompresses and normalizes the GBM vasculature and enhances anti-tumor immunity to improve treatment to ICB, with or without the standard of care. As with many immunotherapy regimens, we observed a differential response to losartan and ICB therapy. Utilizing a bi-hemispheric mouse model for biomarker evaluation, we found that the composition of the immune TME prior to ICB treatment can serve as a predictive biomarker of response.

Accordingly, in one aspect the invention includes a method of reducing immunotherapy induced edema in a patient undergoing immunotherapy comprising administering a composition that reduces MT-MMP-1 and -2 activity in the patients in endothelial cells. One example of a composition that reduces MT-MMP-1 and -2 activity in endothelial cells is Ilomastat.

In another aspect, the invention comprises a method of reducing immunotherapy-induced edema in a patient undergoing immunotherapy comprising administering an angiotensin receptor blocker (ARB) to the patient in an amount effective to reduce the edema. Losartan also reduces MT-MMP-1 and -2 activity in endothelial cells. Losartan is an exemplary angiotensin receptor blocker used in the examples, but other angiotensin receptor blockers can be used, for example valsartan, olmesartan, telmisartan, azilsartan medoxomil, irbesartan, candesartan or eprosartan.

The amount of the compositions administered should be adequate to reduce edema in the patient; ideally the amount should be sufficient to eliminate the edema. The dosing regimen can be determined by the physician etc. The method can be used to treat edemas of the brain in patients undergoing immunotherapy treatment for glioblastoma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 shows that ICB increases GBM vascular leakage and induces brain edema. MR T2-FLAIR images obtained from a recurrent GBM patient (A) before and (B) after 4 months of anti-PD-L1 (MEDI4763; NCT02336165) treatment show increased edema after ICB treatment. In addition to ICB-induced inflammation, this change may be due in part to underlying tumor activity or growth. (C) In mice, anti-PD1 antibody (aPD1) treatment increases edema in GL261 tumors compared to IgG control (as measured by wet-dry weight (i.e., water content) evaluation of tumor tissue; n=5). Multiphoton visualization of the brain vasculature via injected tetramethylrhodamine (TAMRA) labeled albumin imaged through transparent cranial windows in mice bearing GFP+GL261 GBM shows that compared to IgG controls (D) there is increased extravasation in anti-PD1-treated tumors after the third consecutive dose (E). (F) Quantification shows that more albumin in anti-PD1-treated mice has leaked outside of the tumor blood vessels (n=3). (Bar plots: mean±SEM; Student's unpaired t-test; *=p<0.05; ***=p<0.001.)

FIG. 2 shows that losartan prevents ICB-induced edema by downregulating tumor endothelial cell (TEC) MT-MMP-1 and -2 expression. Losartan decreases anti-PD1-induced edema in (A) GL261 and (B) 005 GSC models but not in (C) CT2A after 2 weeks of treatment (n=5-9). (D) scRNASeq of TECs reveals a set of downregulated genes that includes those related to metabolism (e.g., Adh1, Ildr2), angiogenesis/migration (e.g., Cnpy2, Igf1r), solute carriers (e.g., Slc35f2, Slc19a3). When applied as an edema signature, this gene set is upregulated in anti-PD1-treated GL261 tumors compared to other treatment arms as visualized via (E) volcano plot, (F) density plot of edema signature scores (methods described in Materials and Methods) by treatment and (G) mean gene expression heat map of edema signature genes. (H) Specialized MT-MMPs (Mt1, Mt2) are among these genes and are expressed in TECs only from the anti-PD1-treated tumors. (1) The MMP-inhibitor Ilomastat (MMPi) controls anti-PD1-induced edema comparably to losartan in GL261 (n=6). (Edema signature: gene expression units=ln(TP100k+1); log₂FC=fold changes>|2|; adjusted p-value<0.05. Bar plots: mean±SEM; one-way ANOVA with Tukey's post-hoc test; *=p<0.05; **=p<0.01; ***=p<0.001.)

FIG. 3 shows that losartan reprograms the glioblastoma tumor microenvironment. (A) TME-related gene-set enrichment analysis pathways downregulated by losartan treatment compared to control in bulk RNASeq of GL261 tumors (n=3). (B) Differential gene expression confirms these effects in matrix molecules such as collagen, hypoxia-related genes, and immune checkpoints. Intravital OCT imaging (to detect perfused vessels vs. non-perfused areas) shows that compared to PBS-treated controls (C), losartan (D) renders tumor blood vessels less tortuous and improves tumor perfusion (outer dashed line—cranial window border; inner dashed line—tumor area). (Sequencing: FDR—false discovery rate; all FDR q-values<0.20; NES—Normalized Enrichment Score; all adjusted p-values<0.05, FC>|2|. Bar plots: mean±SEM; Student's unpaired t-test; *=p<0.05.)

FIG. 4 shows that losartan promotes anti-tumor immunity in the GBM TME. Applying the human-derived signatures from our previous work (47), losartan is found to enrich microglia-like signatures and downregulate global (A) and “M2-like” (B) Tumor-associated macrophage (TAM) signatures vs. controls as assessed in bulk RNASeq samples from GL261 (n=3). t-SNE plots of flow cytometry data of myeloid populations reveal (C) a diverse and largely immunosuppressive (“M2”) microenvironment in GL261 controls that is (D) reprogrammed by losartan treatment to feature fewer myeloid cells that are polarized for anti-tumor (“M1”) activity (MG—microglia). (E) Losartan increases the ratio of anti- to pro-tumor TAMs, assessed via flow cytometry (n=5-7). Highly suppressive TAM subsets (F) CCR2+ and (G) Arg1+(of CD45hiCD11 b+F4/80+) are downregulated in GL261 tumors implanted in Agtr1a^{-/-} mice compared to those implanted in wildtype (WT) C57Bl/6 mice. (H) scRNASeq of CD8+ T cells reveals heightened Gzmb expression under combined treatment compared to anti-PD1 monotherapy. Losartan+anti-PD1 treatment increases (1) cytotoxic (CTL; CD45+CD3+CD8+ GranzymeB+) to regulatory (Treg; CD45+CD3+CD4+FoxP3+) T cell ratios in the tumor, and effector Granzyme+ CD8 (J, not significant) and CD4 (K) T cells in the cervical lymph nodes. (Sequencing: all FDR q-val<0.25, FC>12|, adjusted p-values<0.05. Flow cytometry: Mann-Whitney unpaired t-test or one-way ANOVA with Tukey's post-hoc test; *=p<0.05.)

FIG. 5 shows that losartan improves survival under anti-PD1 treatment with and without the standard of care. Losartan enhances the survival benefit of anti-PD1 therapy in (A) GL261 and (B) 005 GSC tumor models with 15% and 22% long-term survivors (LTS) respectively, with no detectable tumors via micro-ultrasound imaging through transparent cranial windows for over 100 days (d100). In addition to lack of increased edema in the face of ICB treatment (FIG. 2C), (C) the CT2A model displays only a modest response to anti-PD1 therapy that does not result in long-term survivors nor is improved by the addition of losartan treatment. (D) Long-term surviving mice in the 005 GSC model reject a second tumor inoculation, suggesting the formation of an immune memory response. (E) The GL261 model subjected to standard of care (SOC; F) therapy shows an improvement (G) in response to anti-PD1 (16% long-term survivors) that is tripled (43% long-term survivors) in combination with losartan. (H) Long-term surviving mice in the GL261 standard of care model reject a second tumor rechallenge. (Log-rank Mantel-Cox test; *=p<0.05; **=p<0.01; ***=p<0.001; ****=p<0.0001.)

FIG. 6 shows that the bihemispheric model reveals predictors of response to losartan+anti-PD1 treatment. The bihemispheric mouse model can be used to resect one tumor for biomarker analysis prior to losartan+anti-PD1 treatment which has variable responses in GL261-bearing mice (n=9). (A) Using flow cytometry, immune cells were profiled in individual mice under combinatorial therapy. As indicated by the heat-map z-scores (transformed relative populations of immune cell classes), long-term survivors have distinguished pre-treatment biomarker signatures that indicate strong anti-tumor immunity is present in the tumor prior to therapy. (B) The presence of CD4 T cells and higher ratios of CD8 to regulatory T cells in the GBM TME before therapy initiation are predictive of improved survival, while the presence of T regulatory cells and TAMs are associated with decreased survival, assessed via proportionate hazard models. (P-values derived from univariate Cox regression model; HR— Hazard Ratio; CI—Confidence Interval).

FIG. 7 shows that single-cell RNA sequencing (scRNASeq) reveals a diverse murine GBM TME. scRNASeq profiling of the GL261 GBM TME resulted in (A) uniform manifold approximation and projection (UMAP) visualization of Louvain clustering of all cells identified 12 cell clusters corresponding to (B) 8 distinct cell types by differentially expressed genes (Materials and Methods; centered, units of In(TP100k+1)), including tumor endothelial cells (TECs). (C-E) Copy number alterations as detected by inferCNV (Materials and Methods) with differential gene expression (B) are used to distinguish and annotate (E) malignant and non-malignant clusters.

FIG. 8 shows that murine GBM T cells recapitulate those found in human GBM. (A) UMAP visualization of Louvain clustering (Materials and Methods) of T cells (Cd3d+ and Cd3e+) identified three clusters of T cells. (B) Clusters 0 and 2 are CD4+ T cells and cluster 1 is a CD8+ T cell population. (C) Heatmap of differentially expressed genes (centered, units of In(TP100k+1)) shows Cluster 0 is enriched for T regulatory cell (Treg) markers (Foxp3 and Ctla4), and cluster 1 is enriched for genes such as Cd8a and Gzmk, a marker of cytotoxicity. These results mimic in part the T cell phenotypes characterized from scRNASeq of human GBM.

FIG. 9 shows that CD8 T cells are important mediators of ICB-induced edema. To identify which infiltrating immune cells may contribute to ICB-induced edema, we used T cell depleting antibodies in wildtype mice bearing GBM or grew tumors in TCRα/β knockout mice. (A) Anti-PD1-induced edema in GL261 GBM model is significantly decreased in mice treated with an anti-mouse CD8 depleting antibody or in TCRα/β knockout mice bearing GL261 tumors but is not significantly affected by an anti-CD4 depleting antibody (as compared to anti-PD1 treated wildtype C57Bl/6 mice; n=5-8). (B) Volcano plot (x-axis: In(Fold Change); y-axis: −log 10 (Benjamini Hochberg corrected-p value)) of scRNASeq reveals upregulation of genes linked to lymphocyte-endothelial interactions (e.g., Cxcr6, Anxa1, Litat) in CD8 T cells from anti-PD1-treated tumors vs IgG-treated control tumors.

FIG. 10 shows that losartan reduces hyaluronic acid (HA), solid stress, and PD-L1 expression. (A) CT2A tumors express higher amounts of extracellular matrix (ECM) proteins than GL261 including fibrillar collagens (collagens 1 and 3) and HA. In the GL261 model, 2 weeks of losartan treatment reduces (B) HA levels as measured and quantified via immunofluorescent imaging (n=5), (C) solid stress as measured via 3D micro-ultrasound of tissue (upper dashed line) deformation compared to the normal brain (lower dashed line) reference plane (14) (n=4), and (D) PD-L1 protein expression via immunohistochemistry (n=11-13). (Positive stains quantified as area fractions of total tumor area; Bar plots: mean±SEM; Student's unpaired t-test; *=p<0.05.)

FIG. 11 shows that angiotensin system inhibition usage in treatment-naïve GBM patients is associated with better perfusion and survival. At baseline prior to therapy, perfusion MR image analysis (pMRI) reveals that patients on angiotensin system inhibitors (ASI, n=6 pMRI) like losartan have higher overall blood volume and tumor perfusion than patients not on anti-hypertensives (no ASI, n=30 pMRI). When combined with cediranib, patients on ASI (n=7) have higher overall survival (980 vs 527 days) compared to those not on ASIs (n=33). (Tissue function: non-dimensional relationship between perfusion profiles of tumor vs normal tissue for venous and arterial areas, as quantified by vessel architectural imaging (48). These values are shown only for patients with sufficient pMRI quality data. Box plots: mean±SEM; Student's unpaired t-test; *=p<0.05; Survival: Log-rank Mantel-Cox test.)

FIG. 12 shows that the bihemispheric model predicts individual response to immunotherapy. (A) Identical GBM cells are implanted in each hemisphere under transparent cranial windows (for imaging, B). After initial tumor growth and prior to therapy, one tumor is surgically resected and subjected to TME analysis. Flow cytometry analysis of the resected tumor shows that the number and proportion of non-immune (C, CD45−) vs. immune cells (D, CD45+) in a single resected tumor does not vary significantly whether a single or dual implantation has been conducted (n=8). (E) Mice bearing the remaining second tumor receive systemic combinatorial immunotherapy (losartan+anti-PD1 treatment) and are evaluated for individual and variable survival responses (n=8). They are classified based on survival as non-responders (median survival similar to control mice), responders (mice with improved median survival that eventually succumb), and long-term survivors (mice with non-detectable tumors). (Bar plots: mean±SEM; Student's unpaired t-test; *=p<0.05.)

### DETAILED DESCRIPTION

Immune checkpoint blockers (ICBs) have failed in all phase III glioblastoma trials. Here, we found that ICBs induce cerebral edema in some patients and mice with glioblastoma. Through single-cell RNA sequencing, intravital imaging, and CD8+ T cell blocking studies in mice, we demonstrated that this edema results from an inflammatory response following antiprogrammed death 1 (PD1) antibody treatment that disrupts the blood-tumor barrier. Used in lieu of immunosuppressive corticosteroids, the angiotensin receptor blocker losartan prevented this ICB-induced edema and reprogrammed the tumor microenvironment, curing 20% of mice which increased to 40% in combination with standard of care treatment. Using a bihemispheric tumor model, we identified a “hot” tumor immune signature prior to losartan+anti-PD1 therapy that predicted long-term survival.

Our results are as follows.

### Results

### ICB Treatment Disrupts the GBM Vasculature and Induces Edema.

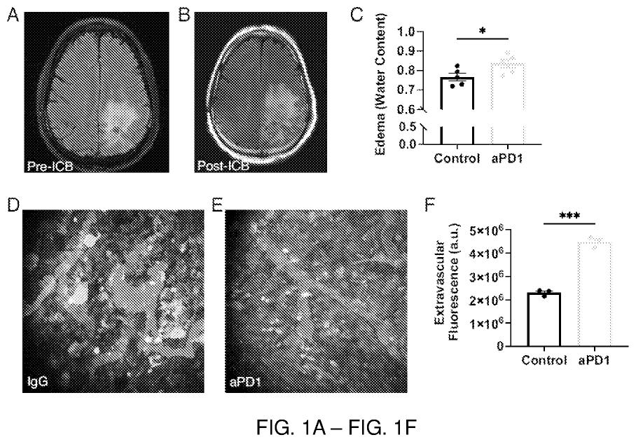

MR imaging revealed ICB-induced edema in some GBM patients (FIG. A, B). In the GL261 model, anti-PD1 antibody treatment recapitulated this increased edema (FIG. 1C). We performed intravital microscopy after injecting the mice with a fluorescent tracer to detect vascular leakage. We found that tumor vessels in control (IgG-treated) mice retained most of the tracer (FIG. 1D), but in anti-PD1-treated mice (FIG. 1E), excess tracer leaked into the surrounding tissue (FIG. 1F), indicating endothelial barrier disruption. Because losartan and other ARBs have been shown to lower vascular endothelial growth factor (VEGF) expression in GBM models and vasogenic edema in retrospective patient studies (3-5), we decided to test the effects of losartan treatment on ICB-induced edema.

We also analyzed our institutional patient cohort of ICB-treated GBM patients to determine the percent increase in the extent of peritumoral edema in the first 6 months post therapy (Table 1). We found that the median percentage increase in edema was 18.8% (−29.6 to 123.5% interquartile range). Factors associated with edema increase included baseline edema volume prior to treatment and radiotherapy treatment; bevacizumab was associated with a decrease in edema. In multivariable Cox regression analysis, neither the patient's baseline edema volume nor their maximum change in edema within 6 months of starting of ICB was associated with overall survival (OS) as measured from the start of ICB treatment (Table 2).

TABLE 1
Clinical factors associated with ICB-induced edema in GBM patients.

| | Univariable linear regression for max % | | | Multivariable linear regression for max % | | |
|---|---|---|---|---|---|---|
| | change in edema within 6 mos of starting ICB | | | change in edema within 6 mos of starting ICB | | |
| | Coefficient | (95% CI) | p value | Coefficient | (95% CI) | p value |
| Age, yr | −1.18/yr | (−5.08-2.71) | 0.55 | −1.10/yr | (−5.01-2.8) | 0.58 |
| Sex | | | | | | |
| Female | ref | | | ref | | |
| Male | 39.70 | (−40.11-119.51) | 0.33 | 30.54 | (−42.12-103.19) | 0.41 |
| KPS | | | | | | |
| <90 | ref | | | ref | | |
| >=90 | 46.82 | (−31.36-125) | 0.24 | 30.49 | (−44.08-105.07) | 0.42 |
| n/a | −81.97 | (−509.04-345.11) | 0.71 | −251.05 | (−646.4-144.31) | 0.21 |
| IDH status | | | | | | |
| Wildtype | ref | | | ref | | |
| Mutant | −88.29 | (−337.38-160.8) | 0.48 | −79.94 | (−325.48-165.6) | 0.52 |
| n/a | 8.77 | (−295.01-312.55) | 0.96 | −70.10 | (−373.57-233.38) | 0.65 |
| MGMT promoter | | | | | | |
| Unmethylated | ref | | | ref | | |
| Methylated | 47.18 | (−33.39-127.75) | 0.25 | 41.92 | (−33.08-116.92) | 0.27 |
| n/a | 97.98 | (−47.45-243.41) | 0.19 | 126.69 | (−24.17-277.54) | 0.10 |
| Radiotherapy | | | | | | |
| No | ref | | | ref | | |
| Yes | 131.04 | (41.26-220.82) | 0.005 | 130.63 | (42.47-218.8) | 0.004 |
| Bevacizumab | | | | | | |
| No | ref | | | ref | | |
| Yes | −121.39 | (−197.16-−45.63) | 0.002 | −92.65 | (−167.85-−17.45) | 0.02 |
| Baseline Enhancing | −2.60E−03/ | (−0.005-−0.0002) | 0.04 | 1.68E−03/ | (−0.0011-0.0044) | 0.23 |
| Volume, mm³ | mm³ | | | mm³ | | |
| Baseline Edema, | −1.58E−03/ | (−0.0022-−0.0009) | <0.001 | −1.39E−03/ | (−0.0022-−0.0006) | <0.001 |
| mm³ | mm³ | | | mm³ | | |

TABLE 2
Multivariable Cox regression analysis of overall
survival¹⁹ among glioblastoma patients that received anti-PD-(L)1.

| | HR | 95% CI | p value |
|---|---|---|---|
| Age, yr | 1.04/yr | (1.01-1.07) | 0.002 |
| Sex | | | |
| Female | ref | | |
| Male | 0.90 | (0.58-1.39) | 0.62 |
| KPS | | | |
| <90 | ref | | |
| >=90 | 0.98 | (0.63-1.52) | 0.93 |
| n/a | 0.84 | (0.1-7.3) | 0.88 |
| IDH status | | | |
| Wildtype | ref | | |
| Mutant | 1.71 | (0.48-6.1) | 0.41 |
| n/a | 1.46 | (0.26-8.19) | 0.67 |
| MGMT promoter | | | |
| Unmethylated | ref | | |
| Methylated | 0.79 | (0.5-1.24) | 0.31 |
| n/a | 1.35 | (0.49-3.72) | 0.56 |
| Radiotherapy | | | |
| No | ref | | |
| Yes | 1.70 | (0.97-2.99) | 0.07 |
| Bevacizumab | | | |
| No | ref | | |
| Yes | 1.89 | (1.2-2.99) | 0.006 |
| Baseline enhancing | 1.00004/mm³ | (1.00002-1.00006) | <0.001 |
| volume, mm³ | | | |
| Baseline edema, mm³ | 1.00000/mm³ | (0.99999-1) | 0.15 |
| Maximum % change | 1.00012/% | (0.99882-1.00143) | 0.85 |
| in edema within | | | |
| 6 months of starting | | | |
| ICB | | | |

Overall survival was measured from initiation of anti-PD(L)1 therapy until death, with censoring at last follow-up. (Patient n = 120, of which 104 died. HR = hazard ratio, CI = confidence interval.)¹⁹

### Losartan Prevents ICB-Induced Edema by Reducing TEC MT-MMP-1 and -2 Expression.

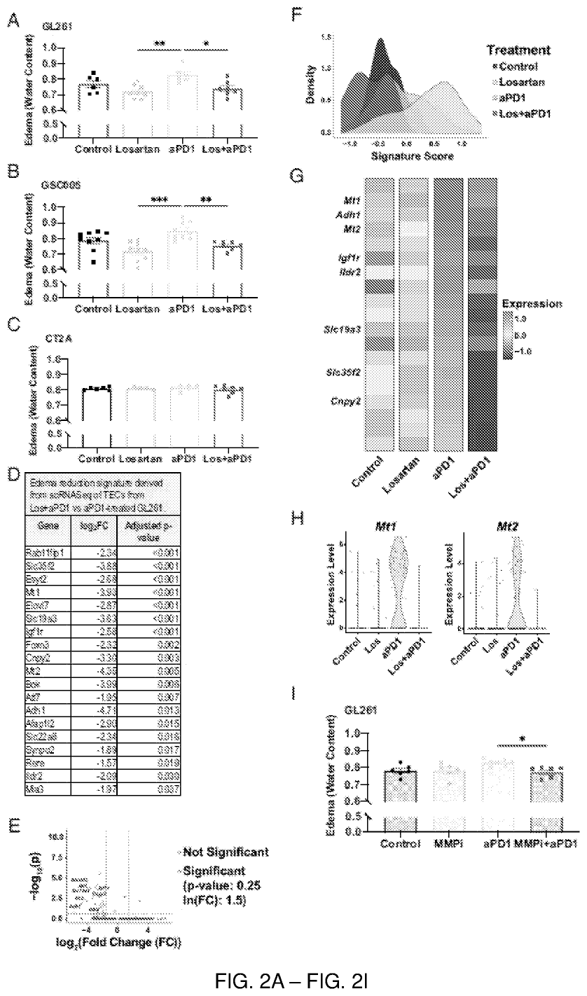

In the GL261 and 005 GSC models (FIG. 2A, B), but not in CT2A (FIG. 2C), we found that anti-PD1 treatment increased edema, while losartan prevented this anti-PD1-induced edema.

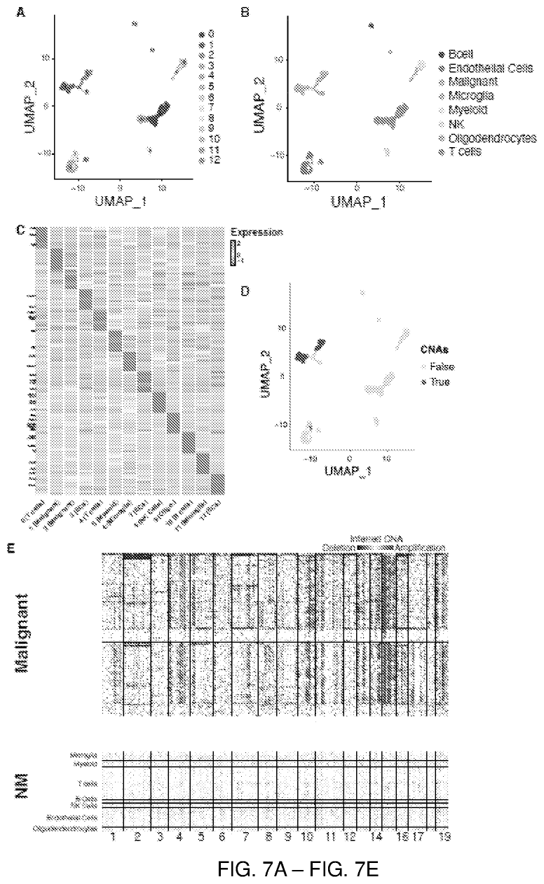

To reveal the edema-reduction mechanism, we performed single cell RNA sequencing (scRNASeq) on TECs in the GL261 model (FIG. 7). We identified a set of genes downregulated in TECs from losartan+anti-PD1-treated tumors vs. anti-PD1 monotherapy (FIG. 2D, E). This edema signature was most highly expressed in TECs from anti-PD1-treated tumors (FIG. 2F, G). Genes included those related to metabolism, angiogenesis/migration, solute carriers, and most notably, a specific subset of MT-MMPs (Mt1 and Mt2, i.e., MMP1 4 and MMP1 5). We did not observe gene expression changes in VEGF/VEGFRs or other known vasogenic edema-related genes in this TEC signature (FIG. 2D, E). Thus, we explored possible inflammatory mechanisms governing ICB-induced edema.

Differentially Expressed Genes from scRNASeq of GL261 Tumors (Data not Shown.)

Anti-PD1 treatment increased edema in the GL261 and 005 GSC (glioma stem cell) models and thus scRNASeq was performed on TECs in the GL261 model to determine the edema-reduction mechanism.

Differentially Expressed Genes from scRNASeq of TECs from GL261 Tumors Under Losartan and/or Anti-PD1 Therapy (Data not Shown).

A set of genes downregulated in TECs from losartan+anti-PD1-treated tumors vs. anti-PD1 monotherapy were identified.

Differentially Expressed Genes from scRNASeq of CD8+ T Cells from GL261 Tumors Under Losartan and/or Anti-PD1 Therapy (Data not Shown).

It was found via scRNASeq that CD8+ T cells are important mediators of ICB-induced edema.

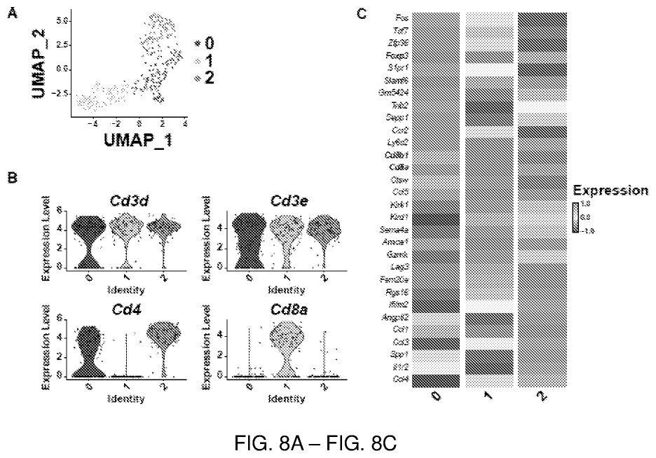

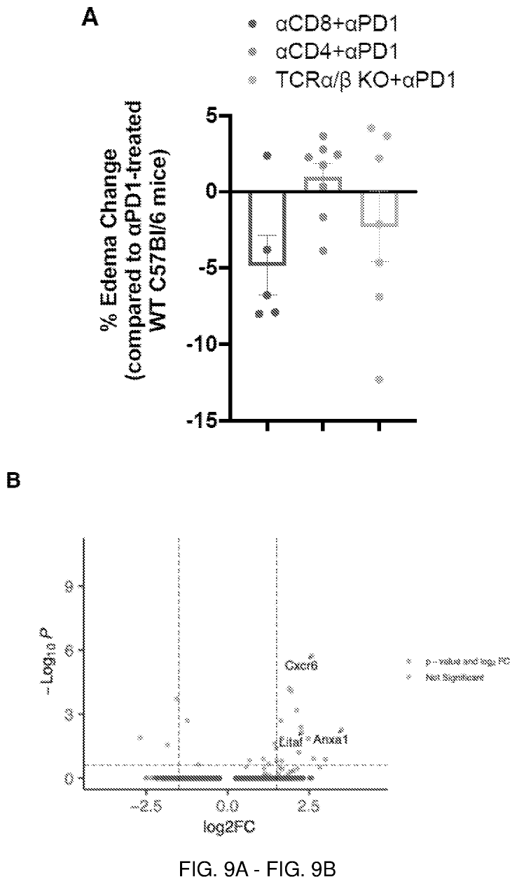

We found via scRNASeq (FIG. 8) and T cell blocking experiments (FIG. 9) that CD8+ T cells are important mediators of ICB-induced edema. Because MMP overexpression in endothelial cells has been linked to blood-brain-barrier (BBB) tight junction disruption and cerebral edema (6, 7), and can be induced by CD8+ T cell interactions (8), we hypothesized that this could be a potential mechanism of ICB-induced edema in GBM. Indeed, Mt1 and Mt2 are only expressed in TECs from anti-PD1-treated tumors (FIG. 2H). To test this mechanism, we gave Ilomastat, a broad spectrum MMP-inhibitor that is non-toxic to GBM cells at physiological levels (9), to mice bearing GL261 tumors under anti-PD1 treatment. We found that Ilomastat phenocopied the ability of losartan to prevent anti-PD1-induced edema (FIG. 21). Because ARBs can modulate other TME features (10-13), we next evaluated the effects of losartan on GBM extracellular matrix (ECM), vasculature, and immune components.

### Losartan Reduces ECM and Solid Stress, Normalizes the Tumor Vasculature, Improves Perfusion, and Decreases Hypoxia and Immunosuppression in GBM.

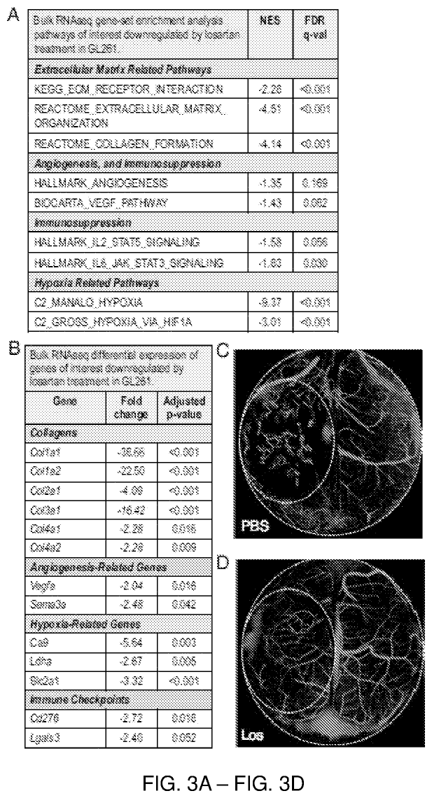

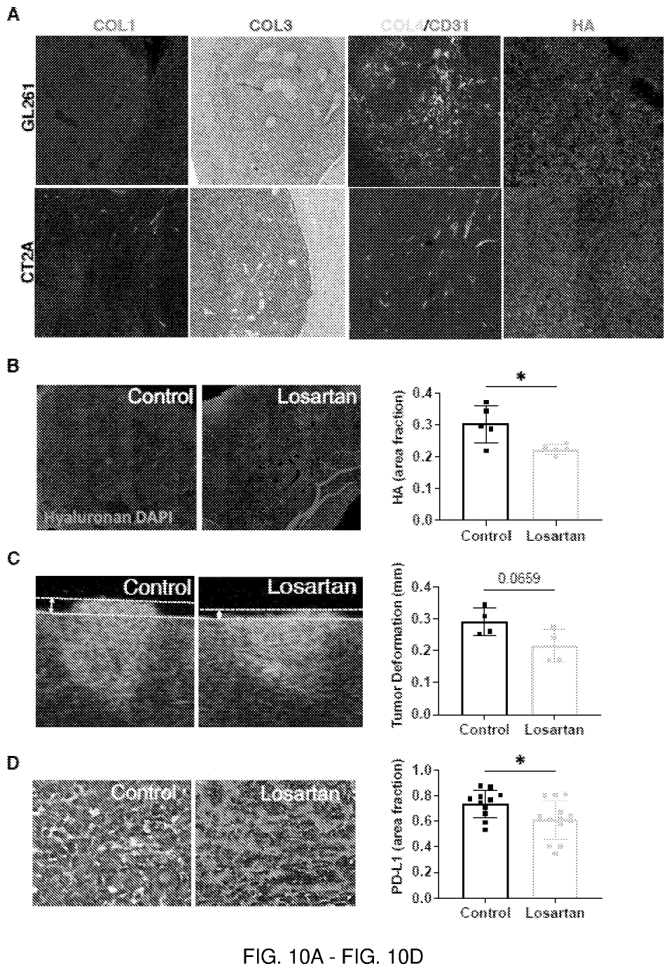

Losartan lowers collagen and hyaluronic acid (HA) levels in extracranial tumors, reducing the physical force “solid stress,” thereby decompressing previously collapsed blood vessels (11). Using bulk RNASeq in GL261, we found that losartan treatment significantly reduced gene expression related to ECM, angiogenesis, immunosuppression, and hypoxia compared to controls (FIG. 3A, B). We observed reduced expression of immune checkpoints both at the transcriptional (FIG. 3B) and protein (FIG. 10) levels. Because HA is a major GBM ECM component, we confirmed via immunohistochemistry that losartan lowers HA levels (FIG. 10). To test if this reduced solid stress, we analyzed tumor tissue deformation (i.e., a measure of solid stress (14)), and found a reduction in losartan-treated tumors (FIG. 10).

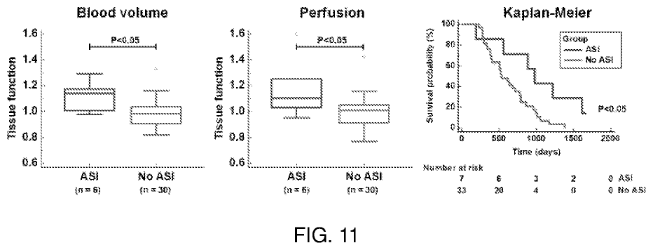

We next determined if losartan improved vascular function in GBM. Using optical coherence tomography (OCT) (15), we found that control tumors featured chaotic abnormal vessels and non-perfused regions (FIG. 3C), whereas losartan-treated tumors had more normalized, straighter, decompressed vessels with greater overall perfusion (FIG. 3D). In perfusion-MR images, we found that GBM patients receiving losartan or other angiotensin system inhibitors also had improved tumor perfusion (FIG. 11).

Losartan repolarizes myeloid cells from pro- to anti-tumor phenotype in GBM.

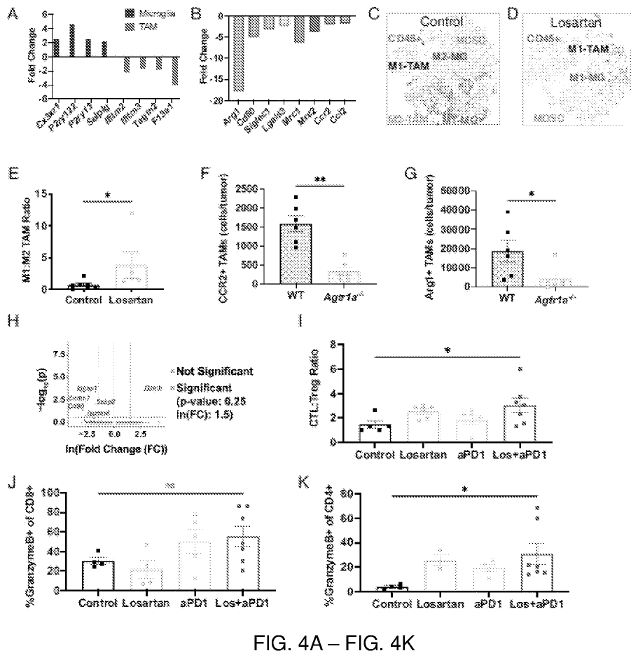

To further explore the beneficial mechanisms of losartan on the TME, we next examined tumor-associated macrophages (TAMs) and resident microglia, as both human and murine GBMs are highly infiltrated by these cells. From bulk RNASeq analyses, we found that losartan upregulated microglia-associated genes (FIG. 4A) and reduced the expression of global (FIG. 4A) and pro-tumor (“M2-like”) TAM-associated genes (FIG. 4B).

Using flow cytometry, we found fewer myeloid cells in losartan-treated tumors with reduced M2-like TAM, microglia, and myeloid-derived suppressor cell (MDSC) compartments (FIG. 4C, D), and an increased ratio of anti-/pro-tumor (“M1-like/M2-like”) TAMs (FIG. 4E). Moreover, pro-tumor TAM populations were significantly reduced in angiotensin type 1 receptor knockout (Agtr1a^{-/-}, i.e., the molecular target of losartan) mice (FIG. 4F, G).

### Losartan Enhances Effector T Cell Function in GBM During ICB Therapy.

Based on the ability of losartan to repolarize the myeloid compartment, we next tested the effects of losartan on T cell function in the face of ICB. We found via scRNASeq that CD8+ T cells from losartan+anti-PD1-treated tumors had higher expression of Gzmb compared to anti-PD1 monotherapy (FIG. 4H). By flow cytometry, we found a significantly increased ratio of cytotoxic Granzyme B+CD8+ T cells to regulatory FoxP3+CD4+ T cells during combined losartan+anti-PD1 treatment (FIG. 4I), as well as an increase in the overall percentages of granzyme B+ effector T cells (CD8, FIG. 4J, and CD4, FIG. 4K) in the draining cervical lymph nodes.

Collectively, our results demonstrated that losartan reprograms the GBM TME from immunosuppressive to immunostimulatory. Thus, we next explored the ability of losartan to enhance survival under ICB therapy.

Losartan Enhances ICB Efficacy without or with the Standard of Care.

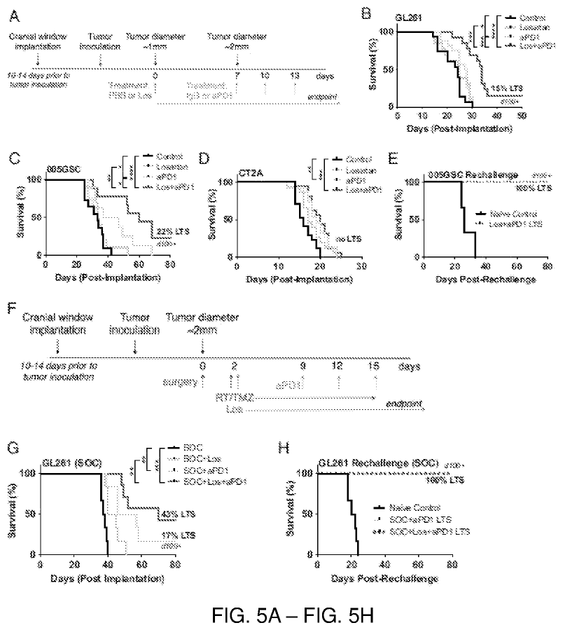

Based on the beneficial TME effects of losartan, we designed our survival studies to administer losartan 7 days prior to and throughout anti-PD1 treatment (FIG. 5A). In GL261 and 005 GSC models, we found that losartan+anti-PD1 antibody doubled animal survival over anti-PD1 monotherapy, and −20% of the mice survived long-term and rejected subsequent tumor re-challenge (FIG. 5B-E). However, in the CT2A model (FIG. 5D), we observed only a modest benefit of anti-PD1 therapy; adding losartan failed to further enhance ICB efficacy. This is not unexpected, given that CT2A has higher ECM content (FIG. 10), is refractory to ICB (16), and did not exhibit increased edema under anti-PD1 treatment (FIG. 2C). In GL261 tumors, we found that standard of care treatment (surgical resection, radiation, and temozolomide; FIG. 5F) enhanced anti-PD1 outcome to produce 16% long-term survivors (FIG. 5G). Long-term survival almost tripled to 43% when losartan was added to standard of care+anti-PD1, and these surviving mice rejected tumor re-challenge (FIG. 5H).

Immune TME Biomarkers from Bihemispheric Tumor Model Predict Individual Response to Iosartan+ICB Therapy.

Because we observed variable responses in individual mice to losartan+anti-PD1 therapy, we sought to identify predictive biomarkers informed by the GBM immune compartment prior to therapy. Building off our recent bilateral breast cancer model (17), we designed a bihemispheric brain tumor model to simultaneously profile immune cells and measure treatment response in individual mice.

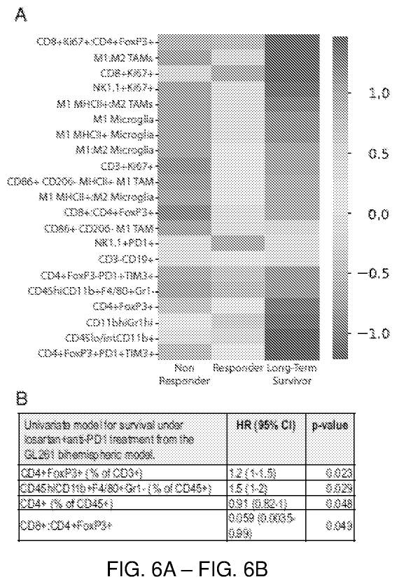

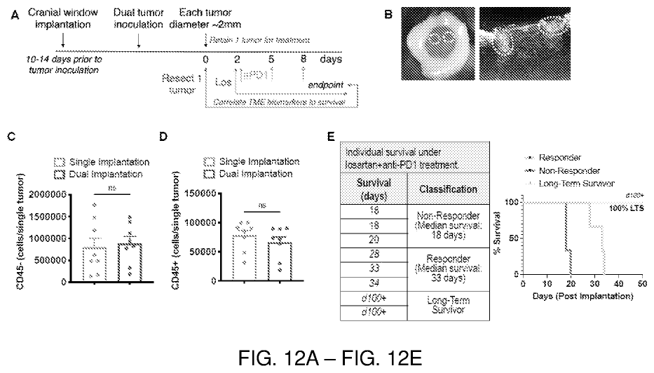

Mice were implanted with two identical GL261 tumors in contralateral hemispheres (FIG. 12). We resected one tumor for biomarker analysis prior to the initiation of losartan+anti-PD1 therapy. Each resected tumor was profiled for immune cells using flow cytometry. Each mouse (now bearing its remaining non-resected tumor) was evaluated for individual response to losartan+anti-PD1 therapy. Mice were classified based on survival as non-responders, responders (improved median survival), and long-term survivors (no detectable tumor) (FIG. 12). We found that, before the initiation of treatment, tumors from long-term surviving mice had strong anti-tumor immune profiles compared to non-responders and responders, including increased ratios of cytotoxic Granzyme B+CD8 T cells to regulatory FoxP3+CD4 T cells, and “M1-like” to “M2-like” TAMs and microglia (FIG. 6A). Immune biomarkers (T regulatory cells, TAMs, CD4 T cells, and cytotoxic to regulatory T cells ratios) were significantly correlated with survival via univariate Cox proportional hazard models (FIG. 6B).

### SUMMARY

Cerebral edema, a hallmark of GBM, is further exacerbated in a fraction of patients under PD1/PD-L1 treatment (1, 2). We sought to identify an agent that could be used in lieu of immunosuppressive corticosteroids—known to compromise ICB efficacy and effector T cell function (18, 19)—to control ICB-induced edema.

Losartan is a small molecule ARB commonly prescribed as an anti-hypertensive agent. Losartan can cross the BBB, and ARB use has been reported to be associated with reduced brain edema and lower steroid dosages in GBM patients undergoing chemoradiation treatment (4, 5, 20, 21). In two syngeneic GBM models, we showed that losartan prevented anti-PD1-induced edema. Brain edema is attributed largely to overexpression of VEGF, which increases vascular permeability (22). However, bevacizumab—an anti-VEGF antibody that can control edema—failed to improve OS in GBM patients under ICB therapy (1), suggesting a VEGF-independent mechanism for ICB-induced edema.

Our sequencing and T cell blockade studies indicate the involvement of inflammatory edema. Using scRNASeq analysis, we derived a signature of edema prevention in TECs that included downregulation of MT-MMP-1 and -2 by losartan. T cell interactions with endothelial cells increase MMP expression (8), which can disrupt tight junctions, leading to a compromised BBB (6). However, MT-MMP-1 and 2 have not yet been linked directly to cerebral edema. Our study demonstrates the role of MMPs in mediating anti-PD1-induced edema, generating a working model that CD8+ T cells infiltrating into the GBM TME in response to ICB, interact with TECs, inducing their increased expression of MT-MMP-1 and -2. This results in a disrupted blood-tumor-barrier and increased edema. Importantly, although losartan can reduce VEGF (3), our results indicate that ICB-induced edema is not VEGF-dependent, but rather due to an inflammatory response.

The immunosuppressive nature of the GBM TME stems from multiple factors. Abnormally high ECM deposition is a key contributor; HA and fibrillar collagens are expressed several-fold higher in GBM than in normal brain tissues (23, 24). This contributes to elevated solid stress that impairs perfusion by compressing tumor blood vessels (25). Reduced perfusion limits tumor oxygenation, drug delivery, and trafficking of anti-tumor immune cells into the GBM TME. This hostile TME contributes to exclusion and exhaustion of CTLs while promoting the infiltration and activation of immunosuppressive Tregs and pro-tumor myeloid cells including TAMs (26, 27). We and others have shown that losartan decreases TGF-3 in mice and cancer patients, thus promoting immune stimulation in non-CNS tumors (11, 12, 28). However, these effects and the underlying mechanisms have not been investigated in GBM.

Our results indicate that losartan repolarizes TAMs and microglia—both of which promote immunosuppression, and are associated with poor prognosis in GBM (29). We recently showed that high expression of the pro-tumor myeloid receptor, C-C chemokine receptor type 2 (CCR2) is associated with poor prognosis in GBM patients, and that targeting CCR2 enhances ICB outcome in GBM models (30). Our results here indicate that angiotensin inhibition not only reduces the presence of CCR2-positive TAMs and other pro-tumor myeloid cells but also reprograms the compartment to an anti-tumor phenotype. In extracranial mouse and human tumors, we have linked losartan (and similar ARBs) to anti-tumor T cell gene expression, presence, and activity (10, 28, 31). Here, we observed improved effector T cell infiltration and function during combined losartan+anti-PD1 therapy. Importantly, although losartan reduces inflammatory responses that contribute to ICB-induced edema, it does not abrogate anti-tumor immune activity.

We have shown that losartan (and similar ARBs) can improve response to cytotoxic and ICB in pancreatic and metastatic breast cancer mouse models, respectively (10, 11). Here, we found in GBM that losartan improves anti-PD1 outcomes in the 005 GSC and GL261 models, but not in CT2A. This could be due in part to excess ECM deposition in CT2A compared to other models, as well as its lack of responsiveness to ICB, and exclusion and exhaustion of CD8 T cells even in the face of anti-PD1 therapy (16, 32). This supposition explains the lack of ICB-induced inflammatory edema in the CT2A model. To lay the groundwork for future clinical translation, we used our recently established standard of care model (16) and further improved the durability of losartan+anti-PD1. The lack of secondary tumor formation after re-challenge in “cured” mice suggests the formation of an immune memory response.

Variable patient response to ICB therapy is a stark and challenging clinical reality. There is an unmet need to identify robust and predictive biomarkers of ICB response, due in part to a lack of mechanistic insight into what drives resistance vs. response. This is particularly the case for GBM patients who present with heterogeneous immune landscapes that may drive variable response to ICB (33-35). Indeed, we observed differential responses within a single treatment arm, even in genetically identical mice bearing tumors grown from the same model and batch of GBM cells.

Building on similar approaches in brain, breast, and subcutaneous sites (17, 36), we developed a bihemispheric tumor model to predict response to losartan+anti-PD1 immunotherapy. Unlike previous studies, however, we utilized this “resection-and-response” approach to evaluate the composition of the GBM immune compartment priorto ICB therapy. Flow cytometry analyses from the bihemispheric model revealed that an immunostimulatory (or “hot”) immune compartment in the TME prior to losartan+anti-PD1 is associated with long-term survivors. This is in line with a recent retrospective transcriptomic analysis showing that patients with “immune-favorable TMEs” benefit the most from immunotherapy (37). This approach allows us to establish predictive biomarkers that could be used to inform selection of GBM patients who may respond to losartan+ICB in future clinical trials based on their tumor immune compartment at the time of surgical resection.

A phase III prospective trial with losartan in GBM failed to improve median OS in combination with the standard of care (38). Similarly, our preclinical results indicate that losartan does not improve OS in GBM mouse models under the standard of care unless it is administered in conjunction with ICB. Retrospective studies (e.g., in non-small cell lung, GI, and GU cancers (39-41)) suggest that patients under angiotensin system inhibitors may have better response to ICB therapy. Losartan is also under clinical testing for ICB combined with cytotoxic therapy in pancreatic ductal adenocarcinoma patients (NCT03563248) following a successful Phase II trial based on our preclinical findings (42).

The above-described results were obtained using the following materials and methods.

### Materials and Methods

### Patient Cohorts

A total of 120 patients with pathologically confirmed World Health Organization CNS grade 4 GBM or astrocytoma were identified that were treated with PD1 or PD-L1 ICB at the time of tumor recurrence from December 2013 to November 2020. The analysis was conducted with Dana-Farber Cancer Institute institutional review board approval (protocol 19-360). Informed consent was obtained in writing from each patient involved in this study prior to their enrollment. The outcome of interest was the percentage of maximum edema increase during the first 6 months following the initiation of ICB. The associations between the outcome and patient clinicopathologic features (including age, sex, KPS (Karnofsky performance score), IDH (isocitrate dehydrogenase) mutation status, MGMT (O(6)-methylguanine-DNA methyltransferase) promoter methylation status, radiotherapy, bevacizumab, baseline enhancing tumor volume, and base line edema) were evaluated using univariable and multivariable linear regression. Two-sided P values <0.05 were considered significant. As a secondary analysis, OS was assessed using multivariable Cox regression. OS was measured from the start of ICB treatment to death and otherwise censored at the last follow-up.

### Cell Culture

Three murine syngeneic cell lines from the C57Bl/6 background were utilized in this study: GL261 (provided by the Frederick National Laboratory, National Cancer Institute), CT2A (provided by Dr. Thomas N. Seyfried, Boston College), and 005 GSC (provided by Dr. Samuel D. Rabkin, Massachusetts General Hospital). Low-passage parental cell stocks—lacking transfection of potentially immunogenic luciferase or fluorescent reporters—were utilized for all studies with one exception: GFP+GL261 cells were used for the multiphoton microscopy of BBB/BTB permeability (described below under “Intravital Imaging”). All cells were subjected to suspension culture techniques to produce neurospheres and were grown in serum-free conditions using the NeuroCult NS-A proliferation kit (Stemcell Technologies). As described below under “Treatment,” commercially available ICB antibodies (from BioXCell) with an IgG2a isotype were utilized. Thus, in contrast to previous preclinical GBM investigations (44), and in line with recent findings from our group (16), all of the cell lines utilized here are resistant to anti-PD1 monotherapy.

### Animal Models

### Mice

C57Bl/6 and Agtr1a^{-/-} mice were obtained from the Edwin L. Steele Laboratories, Massachusetts General Hospital. TCRα/β^{-/-} mice were obtained from Dr. Arlene H. Sharpe's laboratory at the Blavatnik Institute, Harvard Medical School. Male and female mice were used, aged 6-8 weeks at the start of experiments. Animal protocols were approved by and performed in accordance with the Institutional Animal Care and Use Committees (MGH/HMS) and the Association for Assessment and Accreditation of Laboratory Animal Care International.

### Tumor Treatment

Brain tumor implantation in the forebrain (50,000-100,000 cells), cranial window surgery and tumor resection as part of the standard of care treatment regimen were conducted as previously described (16, 43, 45). Mice were allowed to recover for 10-14 days after cranial window surgery prior to tumor implantation, and for 2 days after resection surgery (as part of the standard of care or bihemispheric model) prior to treatment initiation.

When tumors reached 1 mm in diameter (7-10 days post-implantation) mice were treated daily with PBS (control) or losartan (Selleckchem) daily at 60 mg/kg until study endpoint. After 1 week of losartan pre-treatment, mice were treated with IgG (control) or anti-PD1 (BioxCell, RMP1-14) every 3 days for 3 doses at 200 ug/mouse. Standard of care mice received concurrently with losartan: 5 days of consecutive radiotherapy (2 Gy/day) and 10 days of consecutive chemotherapy (temozolomide [TMZ], Selleckchem) at 25 mg/kg. All drugs were injected i.p. For flow cytometry, single-cell RNA sequencing (scRNASeq), intravital imaging, histology, and edema measurements, mice were imaged/sacrificed after the 3^{rd} dose of anti-PD1 and/or 2 weeks of losartan treatment.

### Bihemispheric Model

10-14 days after cranial window surgery, mice are implanted with two identical tumors from the same batch of cells, one in each forebrain hemisphere. Tumor development is monitored via 3D-microultrasound; when each tumor reaches 2 mm in diameter, one tumor is surgically excised. Each excised tumor is subjected to biomarker analysis (in this study, immune profiling of the GBM TME) prior to treatment initiation. 2 days after surgical resection, each mouse bearing its remaining tumor undergoes concurrent losartan+anti-PD1 therapy. At endpoint, mice are classified as non-responders, responders (improved median survival), and long-term survivors (no detectable tumor), and evaluated for predictive biomarkers from the resected pre-treatment tumor. The heatmap of immune cell populations or their ratios (z-score transformed) for each survival classification was generated using the Seaborn 0.9.0 package in the Python language environment. Relative pop The Cox proportional hazard regression models were generated using the “survminer” and “survival” packages in the R platform.

### Flow Cytometry

Single-cell suspensions were prepared from tumors and cervical draining lymph nodes that were isolated and dissected under stereotactic microscope. Cells were stained and processed (on a BD LSRFortessa X-20 Cell analyzer) and analyzed (FlowJo, Tree Star) as previously described (16). The following antibodies from BD Biosciences, EBioscience, and BioLegend were used at 1:200 dilutions: CD45-BV605, CD3-BV785, CD4-BV640, CD8-BV711A, NK1.1-APC; FoxP3-BV421; PD1-PerCP710A; TIGIT-PE Cy7; TIM3-PE; CD19-BV510; K167-FITC; GranzymeB-PE Cy7; CD11 b-BV785; MHCII-BV605; F4/80-PerCP Cy5.5; CX3CR1-APC; CD206-PE CY7; CD86-BV650; CCR2-PE; GR1-AF700.

### Edema Measurements

Edema in the tumor was assessed immediately after animal sacrifice via wet/dry weight analysis to determine the water content as previously described (43).

### Histology and Immunostaining

Brains with tumors were prepped and stained for histology as previously described (45) and imaged on a TissueFAXS (TissueGnostics) slide scanner at the Ragon Institute, Massachusetts Institute of Technology.

### Intravital Imaging

### 3D Micro-Ultrasound

Tumor size was visualized by 3D micro-ultrasound in anesthetized mice through the transparent cranial windows (45). Ultrasound was also used to measure tumor deformation as a readout of solid stress, following previous methods (14).

### Multiphoton Analysis of BBB/BTB Permeability

Multiphoton images were acquired in anesthetized mice through transparent cranial windows using a custom built multiphoton microscope coupled to a mode-locked femtosecond pulsed Ti:Sapphire laser with a Zeiss 25×1.05 NA water dipping objective. The 820 nm multiphoton laser excited fluorescein and TAMRA and the emission was collected using a 535-578 nm and 610-685 nm band pass filter, respectively. Retro-orbital injection of TAMRA-conjugated bovine serum albumin (67 k Da, Invitrogen, 0.1 mL of 10 mg/ml) was performed. In vivo images were acquired 60 min after TAMRA injection. All images were subjected to threshold processing and the extravascular fluorescent intensity was measured using the integrated density measurement function (in ImageJ).

### OCT Imaging of Tumor Perfusion

In vivo imaging of perfused vessels was achieved via a custom-built optical coherence tomography (OCT) system as previously described (15). Mice were anaesthetized and imaged throughout losartan treatment. A depth-resolved profile was generated each day, and the raw tomograms were processed as previously described. Images across multiple days were co-registered using the Scale-invariant feature transform (SIFT) algorithm in ImageJ and Python.

### Single-Cell RNA Sequencing

Processing of Murine GBM Samples for scRNAseq

Following the single-cell suspension techniques of flow cytometry, tumor cells were blocked in 1% bovine serum albumin in phosphate-buffered saline solution (1% BSA/PBS). Cell suspensions were subsequently stained for flow cytometry for 30 min at 4° C. using antibodies specific for CD45 [30F11]-VioBlue from Miltenyi, CD3 [145-2C11]-PE from Biolegends, and CD 31 [MEC 13.3]-PE from BD Biosciences. Cells were washed with cold PBS, and then incubated for 15 min in 1.5 mL of 1% BSA/PBS containing 1 uM calcein AM (Life Technologies) and 0.33 uM TO-PRO-3 iodide (Life Technologies). Sorting was performed with the FACS Aria Fusion Special Order System (Becton Dickinson) using 488 nm (calcein AM, 530/30 filter; CD3-PE, 585/42 filter), 640 nm (TO-PRO-3, 670/14 filter), and 405 nm (CD45-VioBlue, 450/50 filter) lasers. Standard, strict forward scatter height versus area criteria were used to discriminate doublets and gate only singleton cells. Viable cells were identified by staining positive with calcein AM but negative for TO-PRO-3. We sorted individual, viable, CD45+CD3− and CD45+CD3+ immune, and CD45− non-immune single cells into 96-well plates containing cold TCL buffer (QIAGEN) with 1% beta-mercaptoethanol. Plates were frozen on dry ice immediately after sorting and stored at −80° C. prior to whole transcriptome amplification, library preparation and sequencing.

Preparation of scRNA-Seq Libraries

Smart-seq2 whole transcriptome amplification, library construction, and sequencing for malignant cells and microglia were performed as previously published (46, 47). Single cell cDNA and sequencing libraries for T-cells and tumor endothelial cells (TECs) were prepared using the SMART-seq2 protocol with multiple adaptations (19): During the dT annealing step trehalose (1 M) was used instead of water to make up the reaction volume. For the reverse transcription step, Maxima RNaseH-minus RT (200 U/ml) was added at 2 U/ml, water was replaced with trehalose (1 M), and betaine was omitted from the reaction. RT was performed at 50 C for 90 minutes followed by 85 C for 5 minutes. PCR pre-amplification was performed for 21 cycles for T cells and for 22 cycles for endothelial cells.

scRNAseq Data Processing

Sequencing data was processed from raw reads to gene expression matrices, starting with fbcl2fastq (v2.20.0) to generate demultiplexed FASTQ files. Bowtie was used to align the resulting paired-end scRNA-seq reads to the mouse transcriptome (mm10). Gene expression levels were quantified as transcripts-per-million (TPM) by running RSEM (v1.2.19) in paired-end mode. Gene expression levels were quantified as transcripts-per-million (TPM) by running RSEM (v1.2.19) in paired-end mode. Total transcripts per cell were normalized to one-hundred thousand (TP100K), as the estimated the complexity of single-cell libraries prepared by SMART-Seq2 (46). The values were then log-transformed to report gene expression as E=ln(TP100K+1).

Quality Control of scRNA-Seq

A gene was considered to be detected in a given cell if its TP100K was greater than 0. Cells with either less than 1000 or greater than 8,000 unique genes detected were excluded; or if a cell had fewer than 20 housekeeping genes, based on a previously identified gene set (46), it was excluded.

### Cell Type and Cell State Identification

Cell types and (when possible) cell states were identified using the R package Seurat (v4.0.0) (https://github.com/satijalab/seurat). Highly variable genes were selected for clustering analyses using FindVariableGenes, which controls for the inherent relationship between the mean and the variance of gene expression. The following thresholds were used for the mean expression (x) and the variance to mean ratio (y): x.low.cutoff=0.1, x.high.cutoff=7, y.cutoff=0.5. Variable genes were identified within each sample and selected the 1,500 variable genes that were most commonly shared across all samples. The expression of each gene was centered to have a mean of zero using ScaleData and performed Principal Components Analysis (PCA) with RunPCA. Graph-based Louvain clustering was performed on the top 20 principal components using FindClusters, with the resolution parameter set to 0.4 and k for the k-nearest neighbor algorithm set to 30. Differentially expressed genes for each cluster of cells were identified using the t test implemented in FindMarkers while adjusting p-values for multiple hypothesis testing using the Bonferroni correction. Lastly, gene expression and clustering results were visualized on a Uniform Manifold

Approximation and Projection embedding (UMAP) of the top twenty PCs using RunUMAP with the following settings: min_dist=0.5, number of neighbors=30, and distance metric=Euclidean. Cell types were then annotated by considering the cluster's differentially expressed genes.

### Inference of Copy Number Alterations

Default parameters of inferCNV were used to confirm annotation of malignant cell clusters, we as implemented in the R code (see github.com/broadinstitute/infercnv). The clusters annotated as T cells, Endothelial Cells, Myeloid, Microglia, B cells, NK cells, and Oligodendrocytes were used as reference. A subset of the nonmalignant cells were then used as a reference; no CNAs were detected in the non-malignant cells that were not provided as a reference.

CNAs were scored by first defining the overall CNA level of a given cell as the sum of the absolute CNA estimates across all genomic windows. Cells were then identified with the highest overall (top 10%) CNA level and the average CNA profile of these cells was considered as the CNA profile of the sample. Next, the CNA-R-score was computed for each cell using the Spearman correlation coefficient obtained by comparing its CNA profile to the inferred CNA profile of the sample. Cells with a high CNA-R-score (defined as greater than 25%) were considered malignant by the CNA criterion.

### Differential Gene Expression Between Treatments

To explore variability between the expression profiles of cell types given a specific treatment, the FindMarkers function was used to identify differentially expressed genes between cells of two treatment of a given cell type. Volcano plots were generated using the R package EnhancedVolcano (v1.13.2) (see github.com/kevinblighe/EnhancedVolcano). Genes were considered significant with a corrected p-value <0.25 and log 2FC>1.5.

### Edema Signature Score

The level of edema signature score was calculated using AddModuleScore, which calculates the average expression levels of genes in a signature and subtracts from them the average expression levels of control gene sets (46), to examine gene expression signatures within individual cells. The control gene sets were selected to have comparable expression values to the genes in the signature. All genes were placed into 25 bins based on their average expression across all cells. For each gene in a signature, a random set of 10 genes from the same average expression bin as that gene were chosen. This methodology controls for the differences in cell quality and library complexity across single cells.

### Patient Perfusion MRI Data

Perfusion MR imaging (pMRI) data were collected from patients from trial NCT00662506 and analyzed using the previously established vessel architectural imaging technique (48). Briefly, image voxels can be distinguished as arterial or venous-dominated. “Tissue function” parameters are shown that are the ratio for mean blood volume and perfusion values corrected for corresponding levels of normal brain tissue. These values were quantified only from patients with sufficient pMRI quality data. Kaplan-Meier survival comparisons were calculated from the entire dataset of patients on angiotensin system inhibitors (ASI) like losartan, vs. those not (non-ASI).

### Statistical Analysis

Statistics were performed using Prism (GraphPad Software Inc.). Figure legends depict the number of mice used in each experiment (n), the statistical test used, and the visualization (e.g., mean with error bars showing standard error of the mean). Differences with p<0.05 are considered statistically significant.

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

### USES

### Methods of Treatment

The present methods include administration of therapeutically effective amounts of an angiotensin II receptor blocker (ARB) such as losartan, valsartan, olmesartan, telmisartan, azilsartan, medoxomil, irbesartan, candesartan, and eprosartan. In some embodiments, the administration is by mouth (orally). In some embodiments, the administration is local administration, for example, by injection or infusion into or near a tumor, or systemic administration, for example, by intravenous injection or infusion.

The present methods also include administration of therapeutically effective amounts of a membrane-type matrix metalloproteinase inhibitor such as Ilomastat. In some embodiments, Ilomastat is administered intraperitoneally.

The methods described herein include methods for treatment of edema such as brain edema (e.g., cerebral edema). The methods further include treatment of edema using an angiotensin II receptor blocker or a membrane-type matrix metalloproteinase inhibitor in combination with an immune checkpoint blocker. In some embodiments, the angiotensin II receptor blocker or a membrane-type matrix metalloproteinase inhibitor is administered prior to administering the immune checkpoint blocker (e.g., 3 weeks, 2 weeks, 1 week, 6 days, 5 days, 4 days, 3 days, 2 days, or 1 day prior to initiating administering the immune checkpoint blocker). Methods for assessing tumor response and brain/cerebral edema are known. Exemplary methods are found in Chukwueke U N et al. CNS Oncol. 2019 Mar. 1; 8 (1):CNS28. doi: 10.2217/cns-2018-0007. Epub 2019 Feb. 26. PMID: 30806082; PMCID: PMC6499019; Okada H et al. Lancet Oncol. 2015 November; 16 (15):e534-e542. doi: 10.1016/S1470-2045 (15)00088-1. PMID: 26545842; PMCID: PMC4638131; and Chen X et al. Front Oncol. 2021 Jun. 25; 11:679331. doi: 10.3389/fonc.2021.679331. PMID: 34249718; PMCID: PMC8268004.

### Dosing

An “effective amount” is an amount sufficient to affect beneficial or desired results in a subject (e.g., a human having glioblastoma). For example, a therapeutic amount is one that achieves the desired therapeutic effect, e.g., treating glioblastoma, treating brain edema (e.g., cerebral edema), improving vascular function of a glioblastoma, or reprogramming a glioblastoma microenvironment (e.g., from immunosuppressive to immunostimulatory). This amount can be the same or different from a prophylactically effective amount, which is an amount necessary to prevent onset of disease or disease symptoms. An effective amount can be administered in one or more administrations, applications, or dosages. A therapeutically effective amount of a therapeutic compound (i.e., an effective dosage) depends on the therapeutic compounds selected. The compositions can be administered from one or more times per day to one or more times per week, including once every other day. The skilled artisan will appreciate that certain factors may influence the dosage and timing required to effectively treat a subject, including but not limited to the severity of the disease or disorder, previous treatments, the general health and/or age of the subject, and presence of other diseases. Moreover, treatment of a subject with a therapeutically effective amount of the therapeutic compounds described herein can include a single treatment or a series of treatments.

The term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatoiogy), or 2) ameliorating the disease; for example, ameliorating a disease, condition or disorder in an individual who is experiencing or displaying the pathology or symptomatology of the disease, condition or disorder (i.e., reversing the pathology and/or symptomatology).

Treatment includes, but is not limited to, administration of a therapeutic agent or a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated.

Treatment also includes, in some embodiments, one or more of the following circumstances where losartan (or another angiotensin receptor blocker) reduces extracellular matrix, lowers collagen and hyaluronic acid (HA) levels in extracranial tumors, reduces compressive solid stress, decompresses previously collapsed blood vessels, normalizes tumor vasculature, decreases hypoxia and immunosuppression in glioblastoma, prevents or reduces immune checkpoint blocker induced edema by reducing expression of membrane-type matrix metalloproteinase 1 and membrane-type matrix metalloproteinase 2 in tumor endothelial cells, repolarizes myeloid cells from pro- to antitumor phenotype in glioblastoma, or enhances effector T cell function in glioblastoma during immunotherapy.

Also included are “prophylactic” treatments, which can be directed to reducing the rate of progression of the disease or condition being treated, delaying the onset of that disease or condition, or reducing the severity of its onset.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

In some embodiments, an angiotensin II receptor blocker is administered in an oral dosage form (e.g., a tablet, a pill, or a capsule). Exemplary angiotensin II receptor blocker dosing ranges from 10-400 mg daily (e.g., 10, 20, 30, 40, 50, 60, 70, 80, 90, 100, 150, 200, 250, 300, 350, or 400 mg) are useful in the methods described herein, depending on the angiotensin II receptor blocker to be administered.

As used herein, the term “individual”, “patient” or “subject” are used interchangeably, refers to any animal, including mammals, most preferably humans.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. L. Nayak et al., Randomized Phase II and Biomarker Study of Pembrolizumab plus Bevacizumab versus Pembrolizumab Alone for Patients with Recurrent Glioblastoma. Clin Cancer Res 27, 1048-1057 (2021).
2. L. Qin et al., Advanced MRI assessment to predict benefit of anti-programmed cell death 1 protein immunotherapy response in patients with recurrent glioblastoma. Neuroradiology 59, 135-145 (2017).
3. O. Arrieta et al., Blockage of angiotensin II type I receptor decreases the synthesis of growth factors and induces apoptosis in C6 cultured cells and C6 rat glioma. Br J Cancer 92, 1247-1252 (2005).
4. A. F. Carpentier et al., Steroid-sparing effects of angiotensin-II inhibitors in glioblastoma patients. Eur J Neurol 19, 1337-1342 (2012).
5. A. Kourilsky et al., Impact of Angiotensin-II receptor blockers on vasogenic edema in glioblastoma patients. J Neurol 263, 524-530 (2016).
6. R. J. Turner, F. R. Sharp, Implications of MMP9 for Blood Brain Barrier Disruption and Hemorrhagic Transformation Following Ischemic Stroke. Front Cell Neurosci 10, 1-13 (2016).
7. R. M. Jha, P. M. Kochanek, J. M. Simard, Pathophysiology and treatment of cerebral edema in traumatic brain injury. Neuropharmacology 145, 230-246 (2019).
8. S. Mitola, M. Strasly, M. Prato, P. Ghia, F. Bussolino, IL-12 regulates an endothelial cell-lymphocyte network: effect on metalloproteinase-9 production. J Immunol 171, 3725-3733 (2003).
9. S. Yan et al., MMP inhibitor Ilomastat induced amoeboid-like motility via activation of the Rho signaling pathway in glioblastoma cells. Tumour Biol, 16177-16186 (2016).
10. V. P. Chauhan et al., Reprogramming the microenvironment with tumor-selective angiotensin blockers enhances cancer immunotherapy. Proc Natl Acad Sci USA 116, 10674-10680 (2019).
11. V. P. Chauhan et al., Angiotensin inhibition enhances drug delivery and potentiates chemotherapy by decompressing tumour blood vessels. Nat Commun 4, 1-11 (2013).
12. H. Liu et al., Use of Angiotensin System Inhibitors Is Associated with Immune Activation and Longer Survival in Nonmetastatic Pancreatic Ductal Adenocarcinoma. Clin Cancer Res 23, 5959-5969 (2017).
13. M. O'Rawe et al., The Renin-Angiotensin System in the Tumor Microenvironment of Glioblastoma. Cancers (Basel) 13, 1-17 (2021).
14. H. T. Nia et al., Quantifying solid stress and elastic energy from excised or in situ tumors. Nat Protoc 13, 1091-1105 (2018).
15. B. J. Vakoc et al., Three-dimensional microscopy of the tumor microenvironment in vivo using optical frequency domain imaging. Nat Med 15, 1219-1223 (2009).
16. Z. Amoozgar et al., Targeting Treg cells with GITR activation alleviates resistance to immunotherapy in murine glioblastomas. Nat Commun 12, 1-16 (2021).
17. I. X. Chen et al., A bilateral tumor model identifies transcriptional programs associated with patient response to immune checkpoint blockade. Proc Natl Acad Sci USA 117, 23684-23694 (2020).
18. J. B. lorgulescu et al., Concurrent Dexamethasone Limits the Clinical Benefit of Immune Checkpoint Blockade in Glioblastoma. Clin Cancer Res 27, 276-287 (2021).
19. N. D. Mathewson et al., Inhibitory CD161 receptor identified in glioma-infiltrating T cells by single-cell analysis. Cell 184, 1281-1298 e1226 (2021).
20. K. E. Emblem et al., Abstract B12: Adding angiotensin-system inhibitors to anti-angiogenic therapy reduces vasogenic edema in newly diagnosed glioblastomas but not in recurrent disease. Cancer Research 77, B12-B12 (Abstract) (2017).
21. R. J. Hazlewood, Q. Chen, F. K. Clark, J. Kuchtey, R. W. Kuchtey, Differential effects of angiotensin II type I receptor blockers on reducing intraocular pressure and TGFbeta signaling in the mouse retina. PLoS One 13, e0201719 (2018).
22. R. K. Jain, Antiangiogenesis strategies revisited: from starving tumors to alleviating hypoxia. Cancer Cell 26, 605-622 (2014).
23. B. Delpech et al., Hyaluronan and hyaluronectin in the extracellular matrix of human brain tumour stroma. Eur J Cancer 29A, 1012-1017 (1993).
24. I. J. Huijbers et al., A role for fibrillar collagen deposition and the collagen internalization receptor endo 180 in glioma invasion. PLoS One 5, e9808 (2010).
25. H. T. Nia, L. L. Munn, R. K. Jain, Physical traits of cancer. Science 370, (2020).
26. D. Hambardzumyan, D. H. Gutmann, H. Kettenmann, The role of microglia and macrophages in glioma maintenance and progression. Nat Neurosci 19, 20-27 (2016).
27. M. Lim, Y. Xia, C. Bettegowda, M. Weller, Current state of immunotherapy for glioblastoma. Nat Rev Clin Oncol 15, 422-442 (2018).
28. J. Incio et al., Obesity-Induced Inflammation and Desmoplasia Promote Pancreatic Cancer Progression and Resistance to Chemotherapy. Cancer Discov 6, 852-869 (2016).
29. C. C. Poon, S. Sarkar, V. W. Yong, J. J. P. Kelly, Glioblastoma-associated microglia and macrophages: targets for therapies to improve prognosis. Brain 140, 1548-1560 (2017).
30. J. A. Flores-Toro et al., CCR2 inhibition reduces tumor myeloid cells and unmasks a checkpoint inhibitor effect to slow progression of resistant murine gliomas. Proc Natl Acad Sci USA 117, 1129-1138 (2020).
31. D. Jones et al., Solid stress impairs lymphocyte infiltration into lymph-node metastases. Nat Biomed Eng 5, 1426-1436 (2021).
32. J. K. Khalsa et al., Immune phenotyping of diverse syngeneic murine brain tumors identifies immunologically distinct types. Nat Commun 11, 1-14 (2020).
33. K. Aslan et al., Heterogeneity of response to immune checkpoint blockade in hypermutated experimental gliomas. Nat Commun 11, 1-14 (2020).
34. E. Friebel et al., Single-Cell Mapping of Human Brain Cancer Reveals Tumor-Specific Instruction of Tissue-Invading Leukocytes. Cell 181, 1626-1642 e1620 (2020).
35. F. Klemm et al., Interrogation of the Microenvironmental Landscape in Brain Tumors Reveals Disease-Specific Alterations of Immune Cells. Cell 181, 1643-1660 e1617 (2020).
36. R. M. Zemek et al., Bilateral murine tumor models for characterizing the response to immune checkpoint blockade. Nat Protoc 15, 1628-1648 (2020).
37. A. Bagaev et al., Conserved pan-cancer microenvironment subtypes predict response to immunotherapy. Cancer Cell 39, 845-865 (2021).
38. R. Ursu et al., Angiotensin II receptor blockers, steroids and radiotherapy in glioblastoma-a randomised multicentre trial (ASTER trial). An ANOCEF study. Eur J Cancer 109, 129-136 (2019).
39. R. K. Jain et al., Angiotensin Blockade Modulates the Activity of PD1/L1 Inhibitors in Metastatic Urothelial Carcinoma. Clin Genitourin Cancer 19, 540-546 (2021).
40. T. Tozuka et al., Impact of Renin-angiotensin System Inhibitors on the Efficacy of Anti-PD-1/PD-L1 Antibodies in NSCLC Patients. Anticancer Res 41, 2093-2100 (2021).
41. Z. D. Drobni et al., Renin-angiotensin-aldosterone system inhibitors and survival in patients with hypertension treated with immune checkpoint inhibitors. Eur J Cancer 163, 108-118 (2022).
42. J. E. Murphy et al., Total Neoadjuvant Therapy With FOLFIRINOX in Combination With Losartan Followed by Chemoradiotherapy for Locally Advanced Pancreatic Cancer: A Phase 2 Clinical Trial. JAMA Oncol 5, 1020-1027 (2019).
43. T. E. Peterson et al., Dual inhibition of Ang-2 and VEGF receptors normalizes tumor vasculature and prolongs survival in glioblastoma by altering macrophages. Proc Natl Acad Sci USA 113, 4470-4475 (2016).
44. D. A. Reardon et al., Glioblastoma Eradication Following Immune Checkpoint Blockade in an Orthotopic, Immunocompetent Model. Cancer Immunol Res 4, 124-135 (2016).
45. J. Kloepper et al., Ang-2/VEGF bispecific antibody reprograms macrophages and resident microglia to anti-tumor phenotype and prolongs glioblastoma survival. Proc Natl Acad Sci USA 113, 4476-4481 (2016).
46. I. Tirosh et al., Dissecting the multicellular ecosystem of metastatic melanoma by single-cell RNA-seq. Science 352, 189-196 (2016).
47. A. S. Venteicher et al., Decoupling genetics, lineages, and microenvironment in IDH-mutant gliomas by single-cell RNA-seq. Science 355, pii:eaai8478 (2017).
48. K. E. Emblem et al., Vessel architectural imaging identifies cancer patient responders to anti-angiogenic therapy. Nat Med 19, 1178-1183 (2013).

1. A method for treating glioblastoma in a subject, the method comprising administering a therapeutically effective amount of losartan to the subject, thereby treating glioblastoma.

2. The method of claim 1, further comprising administering an antiprogrammed cell death (PD1/PD-L1) therapy.

3. The method of claim 2, wherein the method comprises administering an anti-PD1 antibody or an anti-PD-L1 antibody.

4. The method of claims 2 or 3, wherein losartan in administered prior to antiprogrammed cell death therapy.

5. The method of claims 2 or 3, where in losartan is administered throughout antiprogrammed cell death therapy.

6. A method for treating cerebral edema in a subject having glioblastoma, the method comprising administering a therapeutically effective amount of losartan to the subject, thereby treating cerebral edema.

7. The method of claim 6, wherein the cerebral edema is an immune checkpoint blocker induced edema.

8. The method of claim 7, wherein the method comprises administering losartan prior to administering an immune checkpoint blocker.

9. The method of claim 7, wherein the method comprises administering losartan concomitantly with the immune checkpoint blocker.

10. A method for improving vascular function of a glioblastoma in a subject, the method comprising administering a therapeutically effective amount of losartan to the subject, thereby improving vascular function of the glioblastoma.

11. A method for reprogramming a glioblastoma tumor microenvironment in a subject, the method comprising administering to the subject a therapeutically effective amount of losartan, thereby reprogramming the glioblastoma tumor microenvironment.

12. The method of claim 11, wherein the glioblastoma tumor microenvironment is reprogrammed from immunosuppressive to immunostimulatory.

13. A method for reducing immunotherapy induced brain edema in a subject in need thereof, the method comprising administering an effective amount of an angiotensin receptor blocker to the subject, thereby reducing immunotherapy induced brain edema in the subject.

14. The method of claim 13, wherein the immunotherapy induced brain edema is an immune checkpoint blocker induced edema.

15. The method of claim 14, wherein the immune checkpoint blocker is an anti-PD1 antibody or an anti-PD-L1 antibody.

16. The method of claim 13 or 14, wherein the brain edema is cerebral edema.

17. The method of any one of claims 13-16, wherein the subject has glioblastoma.

18. A method for treating glioblastoma in a subject in need thereof, the method comprising administering an effective amount of an angiotensin receptor blocker to the subject in combination with an immune checkpoint blocker, thereby treating the glioblastoma.

19. The method of any one of claims 13-18, wherein the angiotensin receptor blocker comprises losartan, valsartan, olmesartan, telmisartan, azilsartan, medoxomil, irbesartan, candesartan, or eprosartan.

20. The method of claim 19, wherein the angiotensin receptor blocker is losartan.

21. The method of claim 19 or 20, wherein losartan is administered to the subject in combination with an anti-PD1 antibody.

22. The method of claim 21, wherein losartan is administered to the subject in combination with an anti-PD1 antibody and a standard of care treatment.

23. The method of claim 22, wherein the standard of care treatment includes surgical resection, radiation, or temozolomide.

24. The method of any one of claims 19-23, wherein losartan reduces immunotherapy induced brain edema in the subject by reducing the expression of membrane-type matrix metalloproteinases in tumor endothelial cells.

25. A method for reducing immunotherapy induced brain edema in a subject in need thereof, the method comprising administering an effective amount of a membrane-type matrix metalloproteinase inhibitor to the subject, thereby reducing immunotherapy induced brain edema in the subject.

26. The method of claim 25, wherein the immunotherapy induced brain edema is an immune checkpoint blocker induced edema.

27. The method of claim 26, wherein the immune checkpoint blocker is an anti-PD1 antibody or an anti-PD-L1 antibody.

28. The method of claim 25 or 26, wherein the brain edema is cerebral edema.

29. The method of any one of claims 25-28, wherein the subject has glioblastoma.

30. A method for treating glioblastoma in a subject in need thereof, the method comprising administering an effective amount of a membrane-type matrix metalloproteinase inhibitor to the subject in combination with an immune checkpoint blocker, thereby treating the glioblastoma.

31. The method of any one of claims 24-30, wherein the membrane-type matrix metalloproteinase inhibitor is Ilomastat.

32. The method of any one of claims 24-31, wherein the membrane-type matrix metalloproteinases are membrane-type matrix metalloproteinase 1 and membrane-type matrix metalloproteinase 2.

33. The method of any one of claims 24-32, wherein the membrane-type matrix metalloproteinases are expressed in tumor endothelial cells.

34. The method of claim 31, wherein Ilomastat is administered to the subject in combination with an anti-PD1 antibody.

35. The method of claim 34, wherein Ilomastat is administered to the subject in combination with an anti-PD1 antibody and a standard of care treatment.

36. The method of claim 35, wherein the standard of care treatment includes surgical resection, radiation, or temozolomide.

37. The method of any one of claims 1-36, wherein the subject is a human.

38. A method of reducing immunotherapy induced edema in a patient undergoing immunotherapy, the method comprising administering a composition that reduces membrane-type matrix metalloproteinase 1 and membrane-type matrix metalloproteinase 2 activity in endothelial cells in the patient.

39. The method of claim 38, wherein the composition is Ilomastat.

40. A method of reducing immunotherapy induced edema in a patient undergoing immunotherapy, the methods comprising administering an angiotensin receptor blocker to the patient in an amount effective to reduce the edema.

41. The method of claim 40, wherein the angiotensin receptor blocker comprises losartan, valsartan, olmesartan, telmisartan, azilsartan, medoxomil, irbesartan, candesartan, or eprosartan.

42. The method of claims 38-41, wherein the patient is a human.
